(19) United States
(12) Patent Application Publication
(10) Pub. No.: US 2025/0032568 A1
(43) Pub. Date: Jan. 30, 2025

(54) TRADITIONAL CHINESE MEDICINE COMPOSITION AND TRADITIONAL CHINESE MEDICINE DECOCTION FOR TREATING GASTROESOPHAGEAL REFLUX DISEASE (GERD), AND PREPARATION METHOD AND USE THEREOF

(71) Applicant: AFFILIATED HOSPITAL OF JIANGXI UNIVERSITY OF TRADITIONAL CHINESE MEDICINE, Jiangxi (CN)

(72) Inventors: Qi DAI, Jiangxi (CN); Kunshan CHEN, Jiangxi (CN); Xiang LIU, Jiangxi (CN); Xiaoqi ZHOU, Jiangxi (CN)

(21) Appl. No.: 18/818,809

(22) Filed: Aug. 29, 2024

Related U.S. Application Data
(63) Continuation-in-part of application No. PCT/CN2023/121826, filed on Sep. 27, 2023.

(30) Foreign Application Priority Data
Jul. 29, 2023 (CN) 202310943769.6

(51) Int. Cl. A61K 36/428; A61K 33/26; A61K 35/02; A61K 35/618; A61K 36/481; A61K 36/54; A61K 36/64; A61K 36/65; A61K 36/718; A61K 36/754; A61K 36/808; A61K 36/8965; A61K 36/8966; A61K 36/8968; A61P 1/04

(52) U.S. Cl. CPC A61K 36/428; A61K 33/26; A61K 35/02; A61K 35/618; A61K 36/481; A61K 36/54; A61K 36/64; A61K 36/65; A61K 36/718; A61K 36/754; A61K 36/808; A61K 36/8965; A61K 36/8966; A61K 36/8968; A61P 1/04; A61K 2236/331; A61K 2236/37

(57) ABSTRACT

The present disclosure provides a traditional Chinese medicine composition for treating gastroesophageal reflux disease (GERD), including the following raw materials in parts by weight: 10 parts to 25 parts of Fructus trichosanthis, 10 parts to 30 parts of Radix scrophulariae, 10 parts to 20 parts of Os sepiae, 3 parts to 6 parts of Fructus evodiae, 6 parts to 12 parts of Ramulus cinnamomi, 5 parts to 15 parts of Rhizoma coptidis, 10 parts to 20 parts of Radix paeoniae alba, 10 parts to 20 parts of Radix ophiopogonis, 10 parts to 20 parts of red ochre, 10 parts to 20 parts of calcined Ossa draconis, 10 parts to 20 parts of calcined Concha ostreae, 10 parts to 30 parts of Radix rehmanniae, 10 parts to 20 parts of Bulbus fritillariae thunbergii, 10 parts to 20 parts of Radix asparagi, 10 parts to 30 parts of Radix Astragali.

### CROSS-REFERENCE TO RELATED APPLICATION

The present application is a national stage application of International Patent Application No. PCT/CN2023/121826, filed on Sep. 27, 2023, which claims priority to Chinese Patent Application No. 202310943769.6 filed to the China National Intellectual Property Administration (CNIPA) on Jul. 29, 2023 and entitled “TRADITIONAL CHINESE MEDICINE COMPOSITION AND TRADITIONAL CHINESE MEDICINE DECOCTION FOR TREATING GASTROESOPHAGEAL REFLUX DISEASE (GERD), AND PREPARATION METHOD AND USE THEREOF”, which is incorporated herein by reference in its entirety.

### TECHNICAL FIELD

The present disclosure belongs to the technical field of traditional Chinese medicine preparations, and specifically relates to a traditional Chinese medicine composition and a traditional Chinese medicine decoction for treating gastroesophageal reflux disease (GERD), and a preparation method and use thereof.

### BACKGROUND

Gastroesophageal reflux disease (GERD) is an esophageal motility disorder characterized by symptoms and/or complications due to the reflux of gastric contents into the esophagus. The GERD includes common classic symptoms such as heartburn and reflux, as well as atypical symptoms of chest pain, cough, hoarseness, dysphagia, foreign body sensation in the pharynx and/or complications. These complications are erosion, bleeding, and stenosis of the esophageal mucosa, bleeding, dental caries, ulcers of the mouth and throat, and asthma. GERD is generally misdiagnosed and missed clinically. In addition, GERD is also divided into non-erosive reflux disease (NERD), reflux esophagitis (RE), and Barrett's esophagus based on endoscopic manifestations. The GERD is a disease involving multiple factors, and its main pathophysiological mechanisms include the weakening of anti-reflux barrier and the enhancement of attack factors. A meta-analysis pointed out that the prevalence of GERD was 7.69% in some community populations in China. However, due to changes in the pace of life and dietary structure in modern society, the incidence of GERD in China is gradually increasing.

Currently, diagnostic methods for GERD include clinical diagnosis of typical symptoms and proton pump inhibitor experiments, as well as various detection technologies that reflect GERD and gastroesophageal motility function, such as X-ray examination, radionuclide scan, 24-h esophageal pH monitoring, esophageal multi-channel intraluminal impedance-pH detection, and wireless pH monitoring. Western medicine treats GERD mainly with proton pump inhibitors (PPIs) or potassium-competitive acid blockers (P-CABs). However, long-term use of the PPIs causes many adverse reactions, including: 1) atrophic gastritis and gastric polyps, 2) affecting the absorption of trace elements including calcium ions and magnesium ions, 3) causing osteoporosis in the hips, wrists and other parts, 4) leading to imbalance of the upper jejunum microflora, increasing the chance of Clostridium difficile infection, 5) increasing the chance of community-acquired pneumonia infection, and 6) reducing the efficacy of antiplatelet drugs such as clopidogrel. Moreover, according to the 2021 ESNM/ANMSconsensuspaper: Diagnosis and management of refractory gastro-esophageal reflux disease, as many as 40% of patients with GERD symptoms are not fully responsive to PPI treatment.

### SUMMARY

An objective of the present disclosure is to provide a traditional Chinese medicine composition and a traditional Chinese medicine decoction for treating GERD, and a preparation method and use thereof. In the present disclosure, the traditional Chinese medicine composition and the traditional Chinese medicine decoction each can effectively treat the GERD.

The present disclosure provides a traditional Chinese medicine composition for treating GERD, including the following raw materials in parts by weight: 10 parts to 25 parts of Fructus trichosanthis, 10 parts to 30 parts of Radix scrophulariae, 10 parts to 20 parts of Os sepiae, 3 parts to 6 parts of Fructus evodiae, 6 parts to 12 parts of Ramulus cinnamomi, 5 parts to 15 parts of Rhizoma coptidis, 10 parts to 20 parts of Radix paeoniae Alba, 10 parts to 20 parts of Radix ophiopogonis, 10 parts to 20 parts of red ochre, 10 parts to 20 parts of calcined Ossa Draconis, 10 parts to 20 parts of calcined Concha ostreae, 10 parts to 30 parts of Radix rehmanniae, 10 parts to 20 parts of Bulbus fritillariae thunbergii, 10 parts to 20 parts of Radix asparagi, 10 parts to 30 parts of Radix astragali, 10 parts to 20 parts of vinegar-quenched Carapax et Plastrum testudinis, 10 parts to 15 parts of Radix achyranthis bidentatae, 10 parts to 20 parts of Fructus Aurantii immaturus, 10 parts to 30 parts of Pinelliae rhizoma praeparatum, and 10 parts to 20 parts of Rhizoma atractylodis macrocephalae.

Preferably, the calcined Ossa Draconis, the calcined Concha ostreae, the vinegar-quenched Carapax et Plastrum testudinis, and the red ochre are at a mass ratio of 2:2:1:2.

Preferably, the Bulbus fritillariae thunbergii and the Os sepiae are at a mass ratio of 1:1.

Preferably, the Fructus evodiae and the Rhizoma coptidis are at a mass ratio of 1:2; and the Rhizoma atractylodis macrocephalae and the Fructus aurantii immaturus are at a mass ratio of 1:2.

Preferably, the Radix astragali and the Pinelliae rhizoma praeparatum are at a mass ratio of 3:1; and the Radix ophiopogonis, the Radix scrophulariae, and the Radix rehmanniae are at a mass ratio of 1:1:1.

Preferably, the traditional Chinese medicine composition includes the following raw materials in parts by weight: 10 parts of the Fructus trichosanthis, 10 parts of the Radix scrophulariae, 20 parts of the Os sepiae, 3 parts of the Fructus evodiae, 6 parts of the Ramulus cinnamomi, 6 parts of the Rhizoma coptidis, 10 parts of the Radix paeoniae alba, 10 parts of the Radix ophiopogonis, 20 parts of the red ochre, 20 parts of the calcined Ossa draconis, 20 parts of the calcined Concha ostreae, 10 parts of the Radix rehmanniae, 20 parts of the Bulbus fritillariae thunbergii, 10 parts of the Radix asparagi, 30 parts of the Radix astragali, 10 parts of the vinegar-quenched Carapax et Plastrum testudinis, 10 parts of the Radix achyranthis bidentatae, 20 parts of the Fructus aurantii immaturus, 10 parts of the Pinelliae rhizoma praeparatum, and 10 parts of the Rhizoma atractylodis macrocephalae.

The present disclosure further provides use of the traditional Chinese medicine composition in preparation of a drug for treating GERD.

The present disclosure further provides a preparation method of a traditional Chinese medicine decoction for treating GERD, including the following steps:
1) mixing the calcined Ossa draconis, the calcined Concha ostreae, the vinegar-quenched Carapax et Plastrum testudinis, and the red ochre in the raw materials of the traditional Chinese medicine composition with water to allow first decoction, and filtering a resulting medicinal solution to obtain a first medicinal solution; where the first decoction includes deep decoction and long-term decoction;
2) mixing the remaining raw materials excluding the calcined Ossa draconis, the calcined Concha ostreae, the vinegar-quenched Carapax et Plastrum testudinis, and the red ochre from the raw materials of the traditional Chinese medicine composition, water, and the first medicinal solution to allow second decoction, and filtering a resulting medicinal solution to obtain a second medicinal solution; and
3) subjecting the second medicinal solution to sterilization and encapsulation to obtain the traditional Chinese medicine decoction.

Preferably, a total volume of the calcined Ossa draconis, the calcined Concha ostreae, the vinegar-quenched Carapax et Plastrum testudinis, and the red ochre and a volume of the water are at a ratio of 1:2, while the first decoction is conducted at 100° C. to 125° C. for 30 min to 35 min in step 1).

Preferably, the remaining raw materials and the water are at a volume ratio of 1:1, while the second decoction is conducted at 100° C. to 125° C. for 30 min to 35 min in step 2).

The present disclosure provides a traditional Chinese medicine composition for treating GERD, including the following raw materials in parts by weight: 10 parts to 25 parts of Fructus trichosanthis, 10 parts to 30 parts of Radix scrophulariae, 10 parts to 20 parts of Os sepiae, 3 parts to 6 parts of Fructus evodiae, 6 parts to 12 parts of Ramulus cinnamomi, 5 parts to 15 parts of Rhizoma coptidis, 10 parts to 20 parts of Radix paeoniae alba, 10 parts to 20 parts of Radix ophiopogonis, 10 parts to 20 parts of red ochre, 10 parts to 20 parts of calcined Ossa draconis, 10 parts to 20 parts of calcined Concha ostreae, 10 parts to 30 parts of Radix rehmanniae, 10 parts to 20 parts of Bulbus fritillariae thunbergii, 10 parts to 20 parts of Radix asparagi, 10 parts to 30 parts of Radix astragali, 10 parts to 20 parts of vinegar-quenched Carapax et Plastrum testudinis, 10 parts to 15 parts of Radix achyranthis bidentatae, 10 parts to 20 parts of Fructus aurantii immaturus, 10 parts to 30 parts of Pinelliae rhizoma praeparatum, and 10 parts to 20 parts of Rhizoma atractylodis macrocephalae. In the present disclosure, from the perspective of understanding and treating GERD in traditional Chinese medicine, a new idea of treating GERD is creatively proposed based on the three-dimensional theory of “endogenous wind”. Liver wind internally disturbs the five internal organs, leading to deficiency of Qi and Yin, and obstruction of phlegm and Qi. The combination of dryness and dampness is the specific pathogenesis of GERD, and can be manifested as rising liver and stomach, falling lung qi, and keeping the spleen in the middle burner. The dynamic changes of viscera and qi mechanisms in the upper heart, lower kidney, triple burners, and five internal organs are referred to as the three-dimensional theory of “endogenous wind”. The endogenous wind refers to liver wind, which pushes up phlegm and dampness in the spleen and stomach. The liver wind can invade the heart and lungs, causing deficiency of both qi and yin in the lungs, insufficient heart qi, imbalance of yin and yang, and also disrupts the normal rise and fall of qi in the middle burner of spleen and stomach. The liver wind can even spread to the kidneys, causing triple burner and five internal organs to be damaged, leading to deficiency of the five internal organs, obstruction of phlegm and dampness, and mixed dryness and dampness, thereby complicating the pathogenesis. The traditional Chinese medicine composition is based on the theory of endogenous wind and triple burners. The endogenous wind carries phlegm and dampness and attacks the chest, diaphragm, triple burner, and the five internal organs, leading to a complex pathogenesis of deficiency and excess of the five internal organs, deficiency of both qi and yin, and a mixture of dryness and dampness. The composition acts as a master for the liver and stomach by calming the adverse-rising energy, as a minister for the spleen by drying dampness and reducing phlegm, as an assistant for the lungs and kidneys by nourishing yin and reducing dryness, and as an aid for the heart by replenishing temperature, yang, and calming the nerves. In this way, strengthening the esophageal acid-resistant barrier and regulating gastroesophageal motility disorders are effective in treating the GERD.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 shows a preparation flow chart of the traditional Chinese medicine decoction for treating GERD in Example 1.

### DETAILED DESCRIPTION OF THE EMBODIMENTS

The present disclosure provides a traditional Chinese medicine composition for treating GERD, including the following raw materials in parts by weight: 10 parts to 25 parts of Fructus trichosanthis, 10 parts to 30 parts of Radix scrophulariae, 10 parts to 20 parts of Os sepiae, 3 parts to 6 parts of Fructus evodiae, 6 parts to 12 parts of Ramulus cinnamomi, 5 parts to 15 parts of Rhizoma coptidis, 10 parts to 20 parts of Radix paeoniae alba, 10 parts to 20 parts of Radix ophiopogonis, 10 parts to 20 parts of red ochre, 10 parts to 20 parts of calcined Ossa draconis, 10 parts to 20 parts of calcined Concha ostreae, 10 parts to 30 parts of Radix rehmanniae, 10 parts to 20 parts of Bulbus fritillariae thunbergii, 10 parts to 20 parts of Radix asparagi, 10 parts to 30 parts of Radix astragali, 10 parts to 20 parts of vinegar-quenched Carapax et Plastrum testudinis, 10 parts to 15 parts of Radix achyranthis bidentatae, 10 parts to 20 parts of Fructus aurantii immaturus, 10 parts to 30 parts of Pinelliae rhizoma praeparatum, and 10 parts to 20 parts of Rhizoma atractylodis macrocephalae.

The traditional Chinese medicine composition is based on the theory of endogenous wind and triple burners. The endogenous wind carries phlegm and dampness and attacks the chest, diaphragm, triple burner, and the five internal organs, leading to a complex pathogenesis of deficiency and excess of the five internal organs, deficiency of both qi and yin, and a mixture of dryness and dampness. The composition acts as a master for the liver and stomach by calming the adverse-rising energy, as a minister for the spleen by drying dampness and reducing phlegm, as an assistant for the lungs and kidneys by nourishing yin and reducing dryness, and as an aid for the heart by replenishing temperature, yang, and calming the nerves. In this way, strengthening the esophageal acid-resistant barrier and regulating gastroesophageal motility disorders are effective in treating the GERD.

In the present invention, the monarch drug is composed of calcined Ossa draconis, calcined Concha ostreae, red ochre, and vinegar-quenched Carapax et Plastrum testudinis, which are heavy and sedimentary, so as to calm the adverse-rising energy, replenish yin and subdue yang. Among them, the calcined Ossa draconis calms the nerves, calms the liver, and subdues yang; the calcined Concha ostreae calms the nerves, subdues yang and replenishes yin; the vinegar-quenched Carapax et Plastrum testudinis nourishes yin and subdues yang; the red ochre calms the liver and subdues yang, and calms the adverse-rising energy. In the ministerial drug, the Rhizoma coptidis and Fructus evodiae in combination can clear liver-fire, lower qi, and stop vomiting; the Fructus Aurantii immaturus and Rhizoma atractylodis macrocephalae work together to promote qi and remove fluids, strengthen the spleen and diuresis; the Bulbus fritillariae thunbergii and Os sepiae are combined to resolve phlegm and dissipate stagnation, reduce antacid and relieve pain, relieve restlessness and stop vomiting; the Pinelliae rhizoma praeparatum and Fructus trichosanthis can reduce phlegm and reduce phlegm. Radix rehmanniae, Radix scrophulariae, Radix ophiopogonis, and Radix asparagi are combined as the adjuvant drug to nourish yin and clear away heat, nourish yin and moisturize the lungs, benefit the stomach and promote fluid production, clear the heart and eliminate troubles; the Radix astragali and Ramulus cinnamomi can nourish qi and raise yang, while the Radix paeoniae alba can suppress liver yang, astringe yin, and soften the liver; the Radix achyranthis Bidentatae causes Qi and blood to flow downward and restores the balance of Qi movement.

In the present disclosure, the traditional Chinese medicine composition preferably includes the following raw materials in parts by weight: 10 parts to 25 parts of Fructus trichosanthis, 10 parts to 30 parts of Radix scrophulariae, 10 parts to 20 parts of Os sepiae, 3 parts to 6 parts of Fructus evodiae, 6 parts to 12 parts of Ramulus cinnamomi, 5 parts to 15 parts of Rhizoma coptidis, 10 parts to 20 parts of Radix paeoniae alba, 10 parts to 20 parts of Radix ophiopogonis, 10 parts to 20 parts of red ochre, 10 parts to 20 parts of calcined Ossa draconis, 10 parts to 20 parts of calcined Concha ostreae, 10 parts to 30 parts of Radix rehmanniae, 10 parts to 20 parts of Bulbus fritillariae thunbergii, 10 parts to 20 parts of Radix asparagi, 10 parts to 30 parts of Radix astragali, 10 parts to 20 parts of vinegar-quenched Carapax et Plastrum testudinis, 10 parts to 15 parts of Radix achyranthis bidentatae, 10 parts to 20 parts of Fructus aurantii immaturus, 10 parts to 30 parts of Pinelliae rhizoma praeparatum, and 10 parts to 20 parts of Rhizoma atractylodis macrocephalae.

In the present disclosure, the calcined Ossa draconis, the calcined Concha ostreae, the vinegar-quenched Carapax et Plastrum testudinis, and the red ochre are at a mass ratio of preferably 2:2:1:2.

In the present disclosure, the Bulbus fritillariae thunbergii and the Os sepiae are at a mass ratio of preferably 1:1.

In the present disclosure, the Fructus evodiae and the Rhizoma coptidis are at a mass ratio of preferably 1:2.

In the present disclosure, the Rhizoma atractylodis macrocephalae and the Fructus aurantii immaturus are at a mass ratio of preferably 1:2.

In the present disclosure, the Radix astragali and the Pinelliae rhizoma praeparatum are at a mass ratio of preferably 3:1.

In the present disclosure, the Radix ophiopogonis, the Radix scrophulariae, and the Radix rehmanniae are at a mass ratio of preferably 1:1:1.

In the present disclosure, calcined Ossa draconis and the calcined Concha ostreae are monarch drugs, and the portions should be high; the vinegar-quenched Carapax et Plastrum testudinis is thick, greasy, and relatively expensive; the Bulbus fritillariae thunbergii and Os Sepiae are the components of Wubeisan powder; the Rhizoma coptidis and Fructus evodiae serve as a liver-fire-purging and stomach-regulating pill; the Rhizoma atractylodis macrocephalae and Fructus aurantii immaturus form a Zhizhusan powder; the Radix astragali and the Pinelliae rhizoma praeparatum can promote qi and resolve phlegm, and promoting qi will eliminate the phlegm; and the Radix ophiopogonis, the Radix scrophulariae, and the Radix rehmanniae are the simplification of Zengye Decoction.

In the present disclosure, the raw materials of the traditional Chinese medicine composition each are purchased from the outpatient traditional Chinese medicine pharmacy of the Affiliated Hospital of Jiangxi University of Traditional Chinese Medicine and have been processed.

In the present disclosure, the traditional Chinese medicine composition includes 10 parts to 25 parts by weight of Fructus trichosanthis; the Fructus trichosanthis is: sweet, slightly bitter, cold; this component acts on lung, stomach, and large intestine meridians; efficacy: clearing away heat and eliminating phlegm, widening the chest and dissipating stagnation, moisturizing dryness and laxation; eliminating phlegm, reducing inflammation, inhibiting bacteria, reducing gastric acid concentration, and inhibiting the formation of ulcers. In this prescription, this component mainly clears away heat and eliminates phlegm, widens the chest and moisturizes dryness.

In the present disclosure, the traditional Chinese medicine composition includes 10 parts to 30 parts by weight of Radix scrophulariae; the Radix scrophulariae is: sweet, bitter, salty, slightly cold, and acts on the spleen, stomach, and kidney meridians; efficacy: clearing heat and cooling blood, nourishing yin and reducing fire, detoxifying and dispersing stagnation; antibacterial and other effects. In this prescription, this component mainly nourishes yin and reduces fire.

In the present disclosure, the traditional Chinese medicine composition includes 10 parts to 20 parts by weight of Os sepiae; the Os sepiae is: salty, astringent, warm, and acts on the spleen and kidney meridians; efficacy: astringent and hemostasis, arresting seminal emission and stopping leucorrhea, antacid and pain reliever, dampness and sore astringent; neutralizing gastric acid, changing the pH value of gastric contents, reducing pepsin activity, and promoting ulcer healing. In this prescription, this component mainly acts as an astringent and antacid.

In the present disclosure, the traditional Chinese medicine composition includes 3 parts to 6 parts by weight of Fructus evodiae; the Fructus evodiae is pungent, bitter, hot, and slightly toxic, and acts on the liver, spleen, stomach, and kidney meridians; efficacy: dispersing cold and relieving pain, lowering qi and stopping vomiting, assisting yang and stopping diarrhea; anti-gastric ulcer, inhibiting platelet aggregation and other effects. In this prescription, this component mainly functions to dispel cold and relieve pain, reduce qi and relieve vomiting.

In the present disclosure, the traditional Chinese medicine composition includes 6 parts to 12 parts by weight of Ramulus cinnamomi; the Ramulus cinnamomi is: pungent, sweet, warm, and acts on the lung, heart, and bladder meridians; efficacy: sweating to relieve the surface, dissipating cold and relieving pain, unblocking yang and transforming qi, neutralizing and reducing inverse conditions; promoting gastrointestinal smooth muscle peristalsis, and improving heart function. In this prescription, this component is mainly responsible for dispersing cold and relieving pain, and neutralizing and reducing adverse effects.

In the present disclosure, the traditional Chinese medicine composition includes 5 parts to 15 parts by weight, preferably 6 parts to 10 parts by weight of Rhizoma coptidis; the Rhizoma coptidis is: bitter, cold, and acts on heart, spleen, stomach, liver, gallbladder, and large intestine meridians; efficacy: clearing away heat and drying dampness, purging fire and detoxifying; anti-gastric ulcer, inhibiting gastric juice secretion and other effects. In this prescription, this component mainly clears heat and dries dampness.

In the present disclosure, the traditional Chinese medicine composition includes 10 parts to 20 parts by weight of Radix paeoniae alba; the Radix paeoniae alba is: bitter, sour, slightly cold, and acts on liver and spleen meridians; efficacy: nourishing blood and regulating menstruation, astringing yin and relieving sweating, softening the liver and relieving pain, calming liver yang; regulating gastrointestinal function, analgesics and spasms, and resisting liver damages. In this prescription, this component mainly acts to calm the liver-yang, astringe the yin, and soften the liver.

In the present disclosure, the traditional Chinese medicine composition includes 10 parts to 20 parts by weight of Radix ophiopogonis; the Radix ophiopogonis is: sweet, slightly bitter, slightly cold, and acts on the heart, lung, and stomach meridians; efficacy: nourishing yin and moistening the lungs, benefiting the stomach and promoting fluid production, clearing the heart and removing troubles; enhancing the phagocytic ability of the reticuloendothelial system, increasing peripheral leukocytes, and promoting humoral immunity and cellular immunity. In this prescription, this component mainly functions to nourish yin and moisten the lungs, benefit the stomach and promote fluid production, and clear the heart and relieve troubles.

In the present disclosure, the traditional Chinese medicine composition includes 10 parts to 20 parts by weight of red ochre; the red ochre is: bitter and cold, and acts on the liver, heart, lung, and stomach meridians; efficacy: calming the liver and subduing yang, calming the qi, cooling blood and stopping bleeding; anti-inflammatory, hemostatic, astringent intestinal wall, protecting the mucosal surface, and promoting intestinal peristalsis. In this prescription, this component mainly calms the liver and subdues yang, and calm the adverse-rising energy.

In the present disclosure, the traditional Chinese medicine composition includes 10 parts to 20 parts by weight of calcined Ossa draconis; the calcined Ossa draconis is: sweet, astringent, and flat, and acts on the heart, liver and kidney meridians; efficacy: calming the nerves, soothing the nerves, soothing the liver and subduing yang, astringing and consolidating astringency; suppressing the central nervous system and regulating immunity. In this prescription, this component mainly acts to calm the nerves, calm the liver, and subdue yang.

In the present disclosure, the traditional Chinese medicine composition includes 10 parts to 20 parts by weight of calcined Concha ostreae; the calcined Concha ostreae is: salty, slightly cold, and acts on the liver, gallbladder, and kidney meridians; efficacy: subduing yang and nourishing yin, calming the nerves, softening and dissipating stagnation, astringing and solidifying astringency; antacid and pain relief, anti-liver damage, immune enhancement, and anti-gastric ulcer. In this prescription, this component is responsible for calming the nerves, subduing yang and replenishing yin.

In the present disclosure, the traditional Chinese medicine composition includes 10 parts to 30 parts by weight, preferably 20 parts by weight of Radix rehmanniae; the Radix rehmanniae is: sweet and cold, and acts on the heart, liver, and kidney meridians; efficacy: clearing away heat, cooling blood, and nourishing yin and producing fluid; anti-ulcer, exciting choline and other effects. In this prescription, this component mainly clears away heat, nourishes yin, and produces fluid.

In the present disclosure, the traditional Chinese medicine composition includes 10 parts to 20 parts by weight of Bulbus fritillariae thunbergii; the Bulbus fritillariae thunbergii is: sweet, slightly cold, and acts on the lung, stomach, heart, and gallbladder meridians; efficacy: clearing away heat and resolving phlegm, relieving irritability, and relieving vomiting; reducing phlegm, inhibiting bacteria, and relieving vomiting. In this prescription, this component mainly clears away heat and resolves phlegm, relieves irritability and relieves vomiting.

In the present disclosure, the traditional Chinese medicine composition includes 10 parts to 20 parts by weight of Radix asparagi; the Radix asparagi is: sweet, bitter, cold, and acts on the lung and kidney meridians; efficacy: nourishing yin and moisturizing dryness, clearing the lungs and promoting fluid production; inhibiting lipid peroxidation, increasing the activity of enzymes related to free radical metabolism, and enhancing humoral and cellular immunity. In this prescription, this component mainly nourishes yin and moisturizes dryness, clears the lungs and promotes fluid production.

In the present disclosure, the traditional Chinese medicine composition includes 10 parts to 30 parts by weight, preferably 20 parts by weight of Radix astragali; the Radix astragali is: sweet, slightly warm, and acts on the spleen and lung meridians; efficacy: nourishing qi and raising yang, nourishing the body and strengthening the exterior, diuresis and swelling, promoting body fluid and nourishing blood, relieving stagnation and relieving paralysis, relieving toxins and draining pus, astringing sores and promoting muscle growth; promoting RNA and protein synthesis, protecting liver, enhancing immunity and other effects. In this prescription, this component is responsible for replenishing qi and raising yang.

In the present disclosure, the traditional Chinese medicine composition includes 10 parts to 20 parts by weight of vinegar-quenched Carapax et Plastrum testudinis; the vinegar-quenched Carapax et Plastrum testudinis is: salty, sweet, slightly cold, and acts on the liver, kidneys, and heart meridians; efficacy: nourishing yin and subduing yang, nourishing the kidneys and strengthening bones, nourishing blood and heart, strengthening menstruation and stopping collapse; promoting immunity and adrenocortical functions. In this prescription, this component is responsible for nourishing yin and replenishing yang.

In the present disclosure, the traditional Chinese medicine composition includes 10 parts to 15 parts by weight of Radix achyranthis Bidentatae; the Radix achyranthis Bidentatae is: bitter, sour, sweet, and acts on the liver and kidney meridians; efficacy: removing blood stasis and stimulating menstruation, nourishing liver and kidney, strengthening muscles and bones, diuresis and relieving stranguria, promoting blood downward flow; liver-protecting and cardiotonic properties, enhancing immunity, and anticoagulant. In this prescription, this component mainly nourishes the liver and kidneys and induces blood to flow downward.

In the present disclosure, the traditional Chinese medicine composition includes 10 parts to 20 parts by weight of Fructus aurantii immaturus; the Fructus aurantii immaturus is: bitter, pungent, sour, slightly cold, and acts on the spleen and stomach meridians; efficacy: relieving stagnant Qi and eliminating accumulation, resolving phlegm and dissipating pimples; inhibiting gastrointestinal smooth muscle to relieve intestinal spasm, promoting gastrointestinal motility, and resisting ulcers. In this prescription, this component is mainly responsible for relieving stagnant Qi, eliminating accumulation, resolving phlegm, and dispersing pimples.

In the present disclosure, the traditional Chinese medicine composition includes 10 parts to 30 parts by weight, preferably 20 parts by weight of Pinelliae rhizoma praeparatum; the Pinelliae rhizoma praeparatum is: pungent and warm, and acts on the spleen, stomach, and lung meridians; efficacy: drying dampness and resolving phlegm; expectorant, inhibiting gastric juice secretion, and promoting bile secretion. In this prescription, this component mainly works to dry dampness and resolve phlegm.

In the present disclosure, the traditional Chinese medicine composition includes 10 parts to 20 parts by weight of Rhizoma atractylodis macrocephalae; the Rhizoma atractylodis macrocephalae is: sweet, bitter, warm, and acts on the spleen and stomach meridians; efficacy: tonifying qi and strengthening the spleen, drying dampness and diuresis, antiperspirant, anticoagulation; promoting gastric emptying and small intestine propulsion, preventing and treating experimental gastric ulcer, enhancing cellular immune function and other effects. In this prescription, this component mainly functions to replenish qi, strengthen the spleen, and remove dampness.

In the present disclosure, the traditional Chinese medicine composition includes preferably the following raw materials in parts by weight: 10 parts of the Fructus trichosanthis, 10 parts of the Radix scrophulariae, 20 parts of the Os sepiae, 3 parts of the Fructus evodiae, 6 parts of the Ramulus cinnamomi, 6 parts of the Rhizoma coptidis, 10 parts of the Radix paeoniae alba, 10 parts of the Radix ophiopogonis, 20 parts of the red ochre, 20 parts of the calcined Ossa draconis, 20 parts of the calcined Concha ostreae, 10 parts of the Radix rehmanniae, 20 parts of the Bulbus fritillariae thunbergii, 10 parts of the Radix asparagi, 30 parts of the Radix astragali, 10 parts of the vinegar-quenched Carapax et Plastrum testudinis, 10 parts of the Radix achyranthis bidentatae, 20 parts of the Fructus aurantii immaturus, 10 parts of the Pinelliae rhizoma praeparatum, and 10 parts of the Rhizoma atractylodis macrocephalae.

In an example of the present disclosure, the traditional Chinese medicine composition includes the following raw materials in parts by weight: 10 parts of the Fructus trichosanthis, 10 parts of the Radix scrophulariae, 20 parts of the Os sepiae, 3 parts of the Fructus evodiae, 6 parts of the Ramulus cinnamomi, 6 parts of the Rhizoma coptidis, 10 parts of the Radix paeoniae alba, 10 parts of the Radix ophiopogonis, 20 parts of the red ochre, 20 parts of the calcined Ossa Draconis, 20 parts of the calcined Concha ostreae, 10 parts of the Radix rehmanniae, 20 parts of the Bulbus fritillariae thunbergii, 10 parts of the Radix asparagi, 30 parts of the Radix astragali, 10 parts of the vinegar-quenched Carapax et Plastrum testudinis, 10 parts of the Radix Achyranthis Bidentatae, 20 parts of the Fructus aurantii immaturus, 10 parts of the Pinelliae Rhizoma Praeparatum, and 10 parts of the Rhizoma atractylodis macrocephalae.

In the present disclosure, the traditional Chinese medicine composition has the effects of calming liver wind, lowering stomach qi, resolving phlegm and dampness, and replenishing qi and yin. This composition can effectively treat GERD, and has desirable effect on the typical core symptoms of GERD (acid reflux, heartburn), reflux chest pain, back pain, extraesophageal syndrome (such as reflux cough, reflux laryngitis), and anxiety and insomnia caused by reflux.

The present disclosure further provides use of the traditional Chinese medicine composition in preparation of a drug for treating GERD.

In the present disclosure, a dosage form of the drug preferably includes an oral dosage form; and the oral dosage form preferably includes decoctions or pills.

In the present disclosure, the treatment of GERD includes the treatment of acid reflux and/or heartburn, reflux chest pain, back pain, or extraesophageal syndrome caused by GERD; and the extraesophageal syndrome preferably includes reflux cough or reflux laryngitis and anxiety and insomnia caused by reflux.

In the present disclosure, the treatment of GERD is preferably achieved by enhancing the esophageal acid-resistant barrier and/or regulating gastroesophageal motility disorders.

In the present disclosure, symptom types of the GERD include one or more of liver and stomach heat stagnation syndrome, gallbladder heat invading the stomach, qi stagnation and phlegm obstruction syndrome, blood stasis and collateral obstruction syndrome, middle deficiency and qi inversion syndrome, and spleen deficiency and dampness and heat syndrome.

The present disclosure further provides a preparation method of a traditional Chinese medicine decoction for treating GERD, including the following steps:
1) mixing the calcined Ossa draconis, the calcined Concha ostreae, the vinegar-quenched Carapax et Plastrum testudinis, and the red ochre in the raw materials of the traditional Chinese medicine composition with water to allow first decoction, and filtering a resulting medicinal solution to obtain a first medicinal solution; where the first decoction includes deep decoction and long-term decoction;
2) mixing the remaining raw materials excluding the calcined Ossa draconis, the calcined Concha ostreae, the vinegar-quenched Carapax et Plastrum testudinis, and the red ochre from the raw materials of the traditional Chinese medicine composition, water, and the first medicinal solution to allow second decoction, and filtering a resulting medicinal solution to obtain a second medicinal solution; and
3) subjecting the second medicinal solution to sterilization and encapsulation to obtain the traditional Chinese medicine decoction.

In the present disclosure, the preparation method of the traditional Chinese medicine decoction preferably includes the following steps:
1) mixing the calcined Ossa draconis, the calcined Concha ostreae, the vinegar-quenched Carapax et Plastrum testudinis, and the red ochre in the raw materials of the traditional Chinese medicine composition with water to allow first decoction, and filtering a resulting medicinal solution to obtain a first medicinal solution; where the first decoction includes deep decoction and long-term decoction;
2) mixing the remaining raw materials excluding the calcined Ossa draconis, the calcined Concha ostreae, the vinegar-quenched Carapax et Plastrum testudinis, and the red ochre from the raw materials of the traditional Chinese medicine composition, water, and the first medicinal solution to allow second decoction, and filtering a resulting medicinal solution to obtain a second medicinal solution; and
3) subjecting the second medicinal solution to sterilization and encapsulation to obtain the traditional Chinese medicine decoction.

In the present disclosure, the calcined Ossa draconis, the calcined Concha ostreae, the vinegar-quenched Carapax et Plastrum testudinis, and the red ochre in the raw materials of the traditional Chinese medicine composition are mixed with water to allow first decoction, and a resulting medicinal solution is filtered to obtain a first medicinal solution; where the first decoction includes deep decoction and long-term decoction.

In the present disclosure, a total volume of the calcined Ossa draconis, the calcined Concha ostreae, the vinegar-quenched Carapax et Plastrum testudinis, and the red ochre and a volume of the water are at a ratio of preferably 1:2, while the first decoction is conducted at preferably 100° C. to 125° C. for preferably 30 min to 35 min; and a filter for filtration has a mesh size of 0.15 mm to 0.25 mm, namely 60 mesh to 100 mesh.

In the present disclosure, the remaining raw materials excluding the calcined Ossa draconis, the calcined Concha ostreae, the vinegar-quenched Carapax et Plastrum testudinis, and the red ochre from the raw materials of the traditional Chinese medicine composition, water, and the first medicinal solution are mixed to allow second decoction, and a resulting medicinal solution is filtered to obtain a second medicinal solution.

In the present disclosure, the remaining raw materials and the water are at a volume ratio of preferably 1:1, while the second decoction is conducted at 100° C. to 125° C. for 30 min to 35 min; and a filter for filtration has a mesh size of 0.15 mm to 0.25 mm, namely 60 mesh to 100 mesh.

In the present disclosure, the second medicinal solution is subjected to sterilization and encapsulation to obtain the traditional Chinese medicine decoction. There are no special restrictions on the sterilization and encapsulation methods, and conventional sterilization and encapsulation methods in this field can be used.

In order to further illustrate the present disclosure, the traditional Chinese medicine composition and the traditional Chinese medicine decoction for treating GERD, and the preparation method and the use thereof provided by the present disclosure are described in detail below with reference to the accompanying drawings and examples, but the accompanying drawings and the examples should not be construed as limiting the protection scope of the present disclosure.

### Example 1 a Traditional Chinese Medicine Decoction for Treating GERD

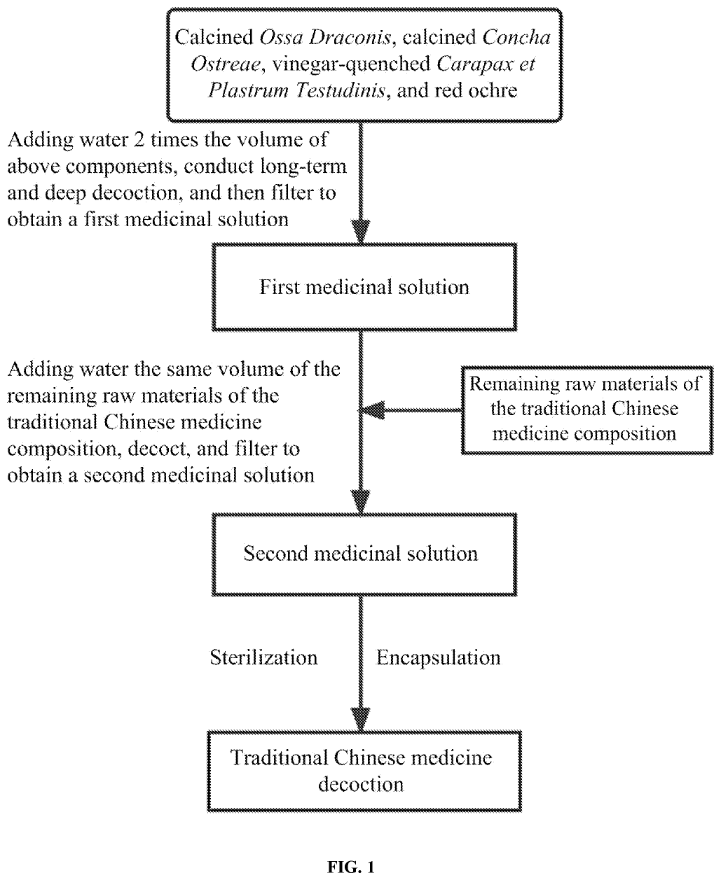

A preparation flow chart was shown in FIG. 1.

The traditional Chinese medicine composition included the following raw materials in parts by weight: 10 parts of the Fructus trichosanthis, 10 parts of the Radix scrophulariae, 20 parts of the Os sepiae, 3 parts of the Fructus evodiae, 6 parts of the Ramulus cinnamomi, 6 parts of the Rhizoma coptidis, 10 parts of the Radix paeoniae alba, 10 parts of the Radix ophiopogonis, 20 parts of the red ochre, 20 parts of the calcined Ossa draconis, 20 parts of the calcined Concha Ostreae, 10 parts of the Radix rehmanniae, 20 parts of the Bulbus fritillariae thunbergii, 10 parts of the Radix asparagi, 30 parts of the Radix astragali, 10 parts of the vinegar-quenched Carapax et Plastrum testudinis, 10 parts of the Radix achyranthis bidentatae, 20 parts of the Fructus aurantii immaturus, 10 parts of the Pinelliae rhizoma praeparatum, and 10 parts of the Rhizoma atractylodis macrocephalae.

A preparation method of the traditional Chinese medicine decoction preferably included the following steps:
1) the calcined Ossa draconis, the calcined Concha ostreae, the vinegar-quenched Carapax et Plastrum testudinis, and the red ochre in the raw materials of the traditional Chinese medicine composition were mixed with water to allow first decoction, and a resulting medicinal solution was filtered with a filter of 60 mesh to 100 mesh to obtain a first medicinal solution; where a total volume of the calcined Ossa draconis, the calcined Concha ostreae, the vinegar-quenched Carapax et Plastrum testudinis, and the red ochre and a volume of the water were at a ratio of 1:2, while the first decoction was conducted at 100° C. to 125° C. for 35 min, and the first decoction included deep decoction and long-term decoction;
2) the remaining raw materials excluding the calcined Ossa draconis, the calcined Concha Ostreae, the vinegar-quenched Carapax et Plastrum testudinis, and the red ochre from the raw materials of the traditional Chinese medicine composition, water, and the first medicinal solution were mixed to allow second decoction, and a resulting medicinal solution was filtered with a filter of 60 mesh to 100 mesh to obtain a second medicinal solution; where the remaining raw materials and the water were at a volume ratio of 1:1, while the second decoction was conducted at 100° C. to 125° C. for 30 min; and
3) subjecting the second medicinal solution to sterilization and encapsulation to obtain the traditional Chinese medicine decoction.

### Example 2

### I. Method

In this example, 80 patients who were diagnosed with GERD in the inpatient and outpatient departments of the Gastroenterology Department of the Affiliated Hospital of Jiangxi University of Traditional Chinese Medicine from 2022.6.1 to 2023.2-28 and met this standard were collected, with a total effective rate of 87.5%.

### (1) Treatment Methods:

Treatment group: 100 mL Bid of the traditional Chinese medicine decoction prepared in Example 1

Control group: 20 mg Bid of an esomeprazole magnesium soluble capsule

### (2) Inclusion Criteria:

Those who met the diagnostic criteria of the “Expert Consensus Opinion on the Diagnosis and Treatment of GERD in Traditional Chinese Medicine (2017)”; (2) those who had no drug contraindications; (3) those who were highly cooperative in treatment and follow-up visits.

### (3) Exclusion Criteria:

1) People with other digestive system diseases; 2) people with malignant tumors; 3) people with severe heart, lung, liver, and kidney diseases; 4) people with upper gastrointestinal disease surgery; 5) pregnancy and lactation patients.

### (4) Diagnostic Criteria in Western Medicine

Those who met the diagnostic criteria for GERD in the “Expert Consensus Opinions on the Diagnosis and Treatment of Traditional Chinese Medicine for GERD (2017)”: clinically, if the patient had typical symptoms of heartburn and acid reflux, he or she could be initially diagnosed as GERD; if upper gastrointestinal endoscopy showed RE and BE manifestations, the diagnosis of this disease was established; for patients suspected of being diagnosed with GERD or those with extraesophageal symptoms suspected to be related to reflux, experimental treatment with PPI could be used. If there was a significant effect, the diagnosis of GERD could generally be established. For those with atypical symptoms, comprehensive analysis including endoscopy, esophageal pH impedance monitoring, and PPI experimental treatment was generally required for diagnosis.

(5) TCM diagnostic criteria: based on the main clinical manifestations such as heartburn, acid reflux, retrosternal burning pain, throat congestion, bitter mouth, belching, nausea and other symptoms, the diagnosis was confirmed through syndrome differentiation.

### (6) TCM Syndrome Differentiation and Classification Criteria:

1) Liver and stomach heat syndrome

Primary symptoms: (1) heartburn; (2) acid reflux.

Secondary symptoms: (1) burning pain behind the sternum; (2) burning pain in the epigastrium; (3) abdominal distension and fullness; (4) belching or reflux; (5) irritability; (6) easy hunger.

Tongue and pulse: (1) red tongue with yellow coating; (2) wiry pulse.

2) Syndrome of gallbladder heat invading the stomach

Primary symptoms: (1) bitter mouth and dry throat; (2) heartburn.

Secondary symptoms: (1) swelling and pain in the flanks; (2) chest and back pain; (3) acid reflux; (4) belching or regurgitation of food; (5) upset and insomnia; (6) easily hungry.

Tongue and pulse: (1) red tongue with yellow and greasy coating; (2) wiry and slippery pulse.

3) Qi stagnation and phlegm obstruction syndrome

Primary symptoms: (1) throat discomfort such as phlegm; (2) chest discomfort.

Secondary symptoms: (1) belching or regurgitation; (2) difficulty swallowing; (3) hoarseness; (4) choking and coughing in the middle of the night.

Tongue and pulse: (1) tongue with white and greasy coating; (2) wiry and slippery pulse.

4) Blood stasis blocking collaterals syndrome

Primary symptom: (1) burning or stabbing pain behind the sternum.

Secondary symptoms: (1) back pain; (2) vomiting blood or melena; (3) heartburn; (4) acid reflux; (5) belching or regurgitation; (6) epigastric tingling.

Tongue and pulse: (1) tongue being dark purple or with ecchymosis; (2) uneven pulse.

5) Moderate deficiency and reverse qi flow syndrome

Primary symptoms: (1) acid reflux or vomiting of water; (2) belching or reflux.

Secondary symptoms: (1) dull epigastric pain; (2) gastric distension; (3) loss of appetite; (4) mental fatigue; (5) loose stools.

Tongue and pulse: (1) Pale tongue with thin coating; (2) thin and weak pulse.

6) Spleen deficiency and dampness-heat syndrome

Primary symptoms: (1) acid reflux after meals; (2) fullness.

Secondary symptoms: (1) burning pain in the epigastrium; (2) chest tightness and discomfort; (3) no desire to eat; (4) tiredness and weakness; (5) loose stools.

Tongue and pulse: (1) pale or red tongue, with thin, yellow, greasy coating; (2) rapid, thin, and slippery pulse.

The above 2 primary symptoms+2 secondary symptoms could be used to diagnose the syndrome with reference to the tongue and pulse. The specific implementation type increased or decreased the dosage according to the syndrome.

### (7) Valid Criteria:

1) Reflux Disease Questionnaire (RDQ)

RDQ is currently the most recognized and widely used diagnostic scale for GERD in the world.

The maximum score for frequency and symptom severity is 40 points, and the GERD is diagnosed when the RDQ score is >12 points.

RDQ scoring criteria: a. Scoring based on the frequency of symptom onset: based on the weekly frequency of four symptoms: heartburn, regurgitation, non-cardiac chest pain, and acid reflux, “never”, “1 week <1 d”, “1 d per 1 week”, “2-3 d per 1 week”, “4-5 d per 1 week”, and “almost every day” were scored as 0, 1, 2, 3, 4, and 5 points, respectively, with a maximum of 20 points. b. Scoring according to the degree of symptom onset: based on the degree of occurrence of the above four symptoms, “Never” scores 0 points; “Symptoms are not obvious, discovered after being reminded by a doctor” scores 1 point; “Symptoms are obvious, affecting daily life, occasionally taking medicine” scores 3 points; “Symptoms are very obvious, affecting daily life, requiring long-term medication” scores 5 points; “Symptoms between 1 and 3” scores 2 points; “Symptoms between 3 and 5” scores 4 points.

2) Esophageal mucosal endoscopy scoring: based on the Los Angeles classification, there are 5 levels. a. Normal: the mucosa is normal without damage (may be accompanied by histological changes); b. Grade A: there is one or more dot-like or strip-like areas of redness and erosion on the mucosa, but the long diameter is <5 mm; c. Grade B: there is one or more punctate or strip-like areas of redness and erosion on the mucosa, with a long diameter >5 mm, but no confluent lesions; d. Grade C: the mucosa has stripes of redness, erosion, and fusion, but has not progressed to full circumference, and the fusion circumference is <75%; e. Grade D: the mucosal lesions are extensive, redness and erosion are fused and cover the entire circumference, and the fusion circumference is >75%. The above grades are scored as 0, 1, 2, 3, and 4, respectively.

3) TCM symptom scoring: referring to the “Guiding Principles for Clinical Research of New Traditional Chinese Medicines” for scoring. The scoring criteria are as follows: primary symptoms including acid reflux, heartburn, belching and reflux; the secondary symptoms including retrosternal burning pain, throat discomfort, bitter mouth, nausea. There are 4 levels according to none, mild, moderate, and severe. The primary symptoms are recorded as 0, 2, 4 and 6 points, respectively, while the secondary symptoms are recorded as 0, 1, 2 and 3 points, respectively.

4) Efficacy criteria: Recovery means the patient's clinical symptoms disappear or basically disappear, the symptom score is >90%, and the gastroscopy shows that the esophageal mucosa returns to normal; Significant effect is that the patient's clinical symptoms improve, the symptom score is reduced by 70% to 94%, and the gastroscopy score is reduced by 2 points; Effective means that patient's clinical symptoms are improved, the symptom score is reduced by 30% to 69%, and the gastroscopy score is reduced by 1 point; Invalid means failure to meet the above standards or worsening; total effective rate=[(Recovery+Significant effect+Effective)/total number of cases]×100%.

### (8) Statistical Method

SPSS26.0 statistical software was used for data analysis. Measurement data were expressed as (X±S), using t test; count data were expressed as [n (%)], using chi-square test. p<0.05 indicated that the difference was statistically significant.

### II. Results:

### (1) General Data Analysis:

### 1) Gender Comparison:

A total of 80 cases that met the criteria were included in this study, and there were no dropouts or discontinuations during the trial. There were 40 patients in the treatment group, including 18 males and 22 females; there were 40 patients in the control group, including 23 males and 17 females. After statistical testing, (P=0.263>0.05) showed that the difference was not statistically significant, and the two experimental groups were comparable in terms of gender, as shown in Table 1.

TABLE 1
Gender comparison of the two groups of patients

| | | Gender | | | |
|---|---|---|---|---|---|
| Group | Number of cases | Male | Female | χ² | P |
| Treatment group | 40 | 18 | 22 | 1.25 | 0.263 |
| Control | 40 | 23 | 17 | | |
| Total | 80 | 41 | 39 | | |

### 2) Age Comparison:

Among the included cases, the youngest in the treatment group was 15 years old, the oldest was 76 years old, and the average age was 45.80±12.69 years old; the youngest in the control group was 27 years old, the oldest was 73 years old, and the average age was 47.63±11.34 years old. After statistical testing, (P=0.284>0.05) showed that the difference was not statistically significant, and the two experimental groups were comparable in terms of age, as shown in Table 2. PGP-22j 1

TABLE 2
Age comparison of the two groups of patients (χ̅ ± S)

| Group | Number of cases | Age/years | t | P |
|---|---|---|---|---|
| Treatment group | 40 | 45.80 ± 12.69 | 0.68 | 0.499 |
| Control | 40 | 47.63 ± 11.34 | | |

### 3) Patient Course Comparison:

In the treatment group, the shortest disease duration was 4 months, the longest was 21 months, and the average disease duration was 11.53±4.47 months; in the control group, the shortest disease duration was 1 month, the longest was 17 months, and the average disease duration was 13.28±5.82 months. After statistical processing, it was found that the differences were not statistically significant (P=0.136>0.05), indicating that the two groups of patients were balanced and comparable, as shown in Table 3.

TABLE 3
Disease course comparison of two groups of patients

| | | Course of disease | | |
|---|---|---|---|---|
| Group | Number of cases | (months) | t | P |
| Treatment group | 40 | 11.53 ± 4.47 | 1.51 | 0.136 |
| Control | 40 | 13.28 ± 5.82 | | |

### 4) RDQ Comparison of the Two Groups of Patients Before Trial

Before the trial, RDQ comparison was conducted on the two groups of patients. After t test, P>0.05, the difference was not statistically significant, indicating that the two groups were comparable, as shown in Table 4.

TABLE 4
RDQ score comparison of two groups before treatment (χ̅ ± S)

| Group | RDQ score | t | P |
|---|---|---|---|
| Treatment group | 22.33 ± 4.44 | 1.88 | 0.061 |
| Control | 20.43 ± 4.62 | | |

### 5) Comparison of Total Clinical Effectiveness of Western Medicine

After the test, among the 40 patients in the treatment group, 8 showed Recovery, 10 showed Significant effect effective, 19 showed Effective, and 3 showed Invalid, with a total effective rate of 92.5%; among the 40 patients in the control group, 4 showed Recovery, 6 showed Significant effect effective, 23 showed Effective, and 7 showed Invalid, with a total effective rate of 82.5%. After testing, P<0.05, the difference was statistically significant, indicating that the treatment method using the traditional Chinese medicine decoction had a clinical efficacy of Western medicine generally better than that simply taking esomeprazole magnesium enteric-coated capsules, as shown in Table 5.

TABLE 5
Efficacy comparison of two groups of patients

| Group | Number of cases | Recovery | Significant effect | Effective | Invalid | Efficacy |
|---|---|---|---|---|---|---|
| Treatment group | 40 | 8 | 10 | 19 | 3 | 92.50% |
| Control | 40 | 4 | 6 | 23 | 7 | 82.50% |

6) Intra-group comparison of RDQ before and after the test in the treatment group. After t test, P<0.05, indicating that the difference was statistically significant. Moreover, the points dropped significantly after the test, indicating that the traditional Chinese medicine decoction could effectively improve the clinical symptoms of patients with Western medicine, as shown in Table 6.

TABLE 6
Comparison of RDQ scores of two groups of patients before and after treatment

| | RDQ score | RDQ score | Intra-group comparison | | Inter-group comparison | |
|---|---|---|---|---|---|---|
| Group | before treatment | after treatment | t | P | t | P |
| Treatment group | 22.33 ± 4.44^{[1]} | 10.85 ± 3.86^{[3]} | 13.35 | P < 0.001^{([1][3])} | 1.88 | 0.061^{([1][2])} |
| Control | 20.43 ± 4.62^{[2]} | 13.73 ± 4.59^{[4]} | 6.51 | P < 0.001^{([2][4])} | 3.04 | 0.030^{([3][4])} |

Notes: [1][3] referred to the scores of the treatment group before and after treatment, respectively, ([1][3]) referred to the P value obtained by comparing the data of these two parts; [2][4] referred to the scores of the control group before and after treatment, respectively, ([2][4]) referred to the P value obtained by comparing the data of these two parts, the same below.

7) Comparison of TCM symptom scores between the two groups of patients after treatment was tested by t test, P<0.05, indicating that the difference was statistically significant. Moreover, the points dropped significantly after the test, indicating that the traditional Chinese medicine decoction could effectively improve the patients' traditional Chinese medicine symptoms, as shown in Table 7.

TABLE 7
Comparison of TCM symptom scores of two groups of patients

| | TCM symptom score | TCM symptom score | Intra-group comparison | | Inter-group comparison | |
|---|---|---|---|---|---|---|
| Group | before treatment | after treatment | t | P | t | P |
| Treatment | 16.38 ± 4.38^{[1]} | 5.98 ± 2.12^{[3]} | 13.52 | P < 0.001^{([1][3])} | 1.55 | 0.124^{([1][2])} |
| group | | | | | | |
| Control | 17.85 ± 4.07^{[2]} | 8.43 ± 4.29^{[4]} | 10.07 | P < 0.001^{([2][4])} | 3.24 | 0.002^{([3][4])} |

### III. Analysis of Typical Clinical Cases

### (1) Case 1

Ms. Li, 38 years old, with a disease duration of 9 months

Symptoms: acid reflux, heartburn, obvious acid breakthrough at night, vomiting of sour water, throat obstruction, belching, stomach noise, restlessness, irritability, red tongue, yellow and greasy coating, wiry, slippery, and slightly rapid pulse.

Diagnosis: Western medicine diagnosis: GERD; TCM diagnosis: esophageal fistula, liver and stomach stagnation and heat type

### Treatment:

100 mL Bid of traditional Chinese medicine decoction

RDQ: 19 points before treatment, 5 points after two weeks of treatment;

Esophageal mucosal score: 2 points before treatment, 0 points after two weeks of treatment;

TCM symptom score: 16 points before treatment, 6 points after two weeks of treatment;

Efficacy: recovery

### (2) Case 2

Ms. Huang, 32 years old, with a disease duration of 7 months

Symptoms: bitter mouth and dry throat, acid reflux and heartburn, fullness and pain in the chest and hypochondrium, belching, upset and insomnia, slightly dry stool, red tongue, yellow and greasy coating, wiry and slippery pulse.

Diagnosis: Western medicine diagnosis: GERD; TCM diagnosis: esophageal fistula, gallbladder heat invading the stomach type

### Treatment:

100 mL Bid of traditional Chinese medicine decoction

RDQ: 18 points before treatment, 6 points after two weeks of treatment;

Esophageal mucosal score: 2 points before treatment, 1 point after two weeks of treatment;

TCM symptom score: 17 points before treatment, 6 points after two weeks of treatment;

Efficacy: significant effect

### (3) Case 3

Ms. Liu, 24 years old, with a disease duration of 4 months

Symptoms: acid reflux, heartburn, throat congestion, chest tightness, sighing, depression, silent inability to eat, white and greasy tongue coating, wiry and slippery pulse.

Diagnosis: Western medicine diagnosis: GERD; TCM diagnosis: esophageal fistula, qi stagnation and phlegm obstruction type

### Treatment:

100 mL Bid of traditional Chinese medicine decoction

RDQ: 15 points before treatment, 5 points after two weeks of treatment;

Esophageal mucosal score: 2 points before treatment, 1 point after two weeks of treatment;

TCM symptom score: 15 points before treatment, 4 points after two weeks of treatment;

Efficacy: significant effect

### (4) Case 4

Ms. Zhou, 56 years old, with a disease duration of 22 months

Symptoms: acid reflux, heartburn, stabbing pain behind the sternum, which persists even at night, throat obstruction, dark purple tongue, deep and uneven pulse

Diagnosis: Western medicine diagnosis: GERD; TCM diagnosis: esophageal fistula, blood stasis and collateral obstruction type

### Treatment:

100 mL Bid of traditional Chinese medicine decoction

RDQ: 16 points before treatment, 8 points after two weeks of treatment;

Esophageal mucosal score: 3 points before treatment, 2 point after two weeks of treatment;

TCM symptom score: 15 points before treatment, 7 points after two weeks of treatment;

Efficacy: effective

### (5) Case 5

Mr. Zhao, 38 years old, with a disease duration of 12 months

Symptoms: acid reflux, belching, conscious feeling of gas rushing up the throat, distended stomach, loss of appetite, fatigue, and loose stools. Pale tongue, thin coating, deep and thin pulse.

Diagnosis: Western medicine diagnosis: GERD; TCM diagnosis: esophageal fistula with deficiency and qi inversion type

### Treatment:

100 mL Bid of traditional Chinese medicine decoction

RDQ: 14 points before treatment, 6 points after two weeks of treatment;

Esophageal mucosal score: 2 points before treatment, 1 point after two weeks of treatment;

TCM symptom score: 16 points before treatment, 6 points after two weeks of treatment;

Efficacy: significant effect

### (6) Case 6

Mr. Wang, 26 years old, with a disease duration of 5 months

Symptoms: burning pain in the epigastrium, noisy pantothenic acid, dry mouth and bitter taste, unwillingness to drink when thirsty, sweet and sticky mouth, sour water when eating sweets, nausea and vomiting, heavy body and tired limbs, yellow urine, difficulty in defecation, yellow and greasy tongue coating, slippery and rapid pulse.

Diagnosis: Western medicine diagnosis: GERD; TCM diagnosis: esophageal fistula, spleen and stomach dampness-heat type

### Treatment:

100 mL Bid of traditional Chinese medicine decoction

RDQ: 15 points before treatment, 6 points after two weeks of treatment;

Esophageal mucosal score: 2 points before treatment, 1 point after two weeks of treatment;

TCM symptom score: 17 points before treatment, 6 points after two weeks of treatment;

Efficacy: significant effect.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1.-20. (canceled)

21. A traditional Chinese medicine composition for treating gastroesophageal reflux disease (GERD), comprising the following raw materials in parts by weight: 10 parts to 25 parts of Fructus trichosanthis, 10 parts to 30 parts of Radix scrophulariae, 10 parts to 20 parts of Os sepiae, 3 parts to 6 parts of Fructus evodiae, 6 parts to 12 parts of Ramulus cinnamomi, 5 parts to 15 parts of Rhizoma coptidis, 10 parts to 20 parts of Radix paeoniae alba, 10 parts to 20 parts of Radix ophiopogonis, 10 parts to 20 parts of red ochre, 10 parts to 20 parts of calcined Ossa draconis, 10 parts to 20 parts of calcined Concha ostreae, 10 parts to 30 parts of Radix rehmanniae, 10 parts to 20 parts of Bulbus fritillariae thunbergii, 10 parts to 20 parts of Radix asparagi, 10 parts to 30 parts of Radix astragali, 10 parts to 20 parts of vinegar-quenched Carapax et Plastrum testudinis, 10 parts to 15 parts of Radix achyranthis bidentatae, 10 parts to 20 parts of Fructus aurantii immaturus, 10 parts to 30 parts of Pinelliae rhizoma praeparatum, and 10 parts to 20 parts of Rhizoma atractylodis macrocephalae.

22. The traditional Chinese medicine composition according to claim 21, wherein the calcined Ossa draconis, the calcined Concha ostreae, the vinegar-quenched Carapax et Plastrum testudinis, and the red ochre are at a mass ratio of 2:2:1:2.

23. The traditional Chinese medicine composition according to claim 21, wherein the Bulbus fritillariae thunbergii and the Os sepiae are at a mass ratio of 1:1.

24. The traditional Chinese medicine composition according to claim 21, wherein the Fructus evodiae and the Rhizoma coptidis are at a mass ratio of 1:2; and the Rhizoma atractylodis macrocephalae and the Fructus aurantii immaturus are at a mass ratio of 1:2.

25. The traditional Chinese medicine composition according to claim 21, wherein the Radix astragali and the Pinelliae rhizoma praeparatum are at a mass ratio of 3:1; and the Radix ophiopogonis, the Radix scrophulariae, and the Radix Rehmanniae are at a mass ratio of 1:1:1.

26. The traditional Chinese medicine composition according to claim 21, comprising the following raw materials in parts by weight: 10 parts of the Fructus trichosanthis, 10 parts of the Radix scrophulariae, 20 parts of the Os sepiae, 3 parts of the Fructus evodiae, 6 parts of the Ramulus cinnamomi, 6 parts of the Rhizoma coptidis, 10 parts of the Radix paeoniae alba, 10 parts of the Radix ophiopogonis, 20 parts of the red ochre, 20 parts of the calcined Ossa draconis, 20 parts of the calcined Concha ostreae, 10 parts of the Radix rehmanniae, 20 parts of the Bulbus fritillariae thunbergii, 10 parts of the Radix asparagi, 30 parts of the Radix astragali, 10 parts of the vinegar-quenched Carapax et Plastrum testudinis, 10 parts of the Radix achyranthis bidentatae, 20 parts of the Fructus aurantii immaturus, 10 parts of the Pinelliae rhizoma praeparatum, and 10 parts of the Rhizoma atractylodis macrocephalae.

27. A preparation method of a traditional Chinese medicine decoction for treating GERD, comprising the following steps:
1) mixing the calcined Ossa draconis, the calcined Concha ostreae, the vinegar-quenched Carapax et Plastrum Testudinis, and the red ochre in the raw materials of the traditional Chinese medicine composition according to claim 1 with water to allow first decoction, and filtering a resulting medicinal solution to obtain a first medicinal solution; wherein the first decoction comprises deep decoction and long-term decoction;
2) mixing the remaining raw materials excluding the calcined Ossa draconis, the calcined Concha ostreae, the vinegar-quenched Carapax et Plastrum testudinis, and the red ochre from the raw materials of the traditional Chinese medicine composition, water, and the first medicinal solution to allow second decoction, and filtering a resulting medicinal solution to obtain a second medicinal solution; and
3) subjecting the second medicinal solution to sterilization and encapsulation to obtain the traditional Chinese medicine decoction.

28. The preparation method according to claim 27, wherein a total volume of the calcined Ossa draconis, the calcined Concha ostreae, the vinegar-quenched Carapax et Plastrum testudinis, and the red ochre and a volume of the water are at a ratio of 1:2, while the first decoction is conducted at 100° C. to 125° C. for 30 min to 35 min in step 1).

29. The preparation method according to claim 27, wherein the remaining raw materials and the water are at a volume ratio of 1:1, while the second decoction is conducted at 100° C. to 125° C. for 30 min to 35 min in step 2).

30. A traditional Chinese medicine decoction prepared by the preparation method according to claim 27.

31. A method for treating GERD, comprising:
subjecting a patient with the GERD to oral administration of the traditional Chinese medicine decoction according to claim 30.

32. The method according to claim 31, wherein the traditional Chinese medicine decoction is applied at an oral dose of 100 milliliter-bis in die (mL Bid).

33. The preparation method according to claim 27, wherein the calcined Ossa draconis, the calcined Concha ostreae, the vinegar-quenched Carapax et Plastrum testudinis, and the red ochre are at a mass ratio of 2:2:1:2.

34. The preparation method according to claim 27, wherein the Bulbus fritillariae thunbergii and the Os sepiae are at a mass ratio of 1:1.

35. The preparation method according to claim 27, wherein the Fructus evodiae and the Rhizoma coptidis are at a mass ratio of 1:2; and the Rhizoma atractylodis macrocephalae and the Fructus aurantii immaturus are at a mass ratio of 1:2.

36. The preparation method according to claim 27, wherein the Radix astragali and the Pinelliae rhizoma praeparatum are at a mass ratio of 3:1; and the Radix ophiopogonis, the Radix scrophulariae, and the Radix rehmanniae are at a mass ratio of 1:1:1.

37. The preparation method according to claim 27, comprising the following raw materials in parts by weight: 10 parts of the Fructus trichosanthis, 10 parts of the Radix scrophulariae, 20 parts of the Os sepiae, 3 parts of the Fructus evodiae, 6 parts of the Ramulus cinnamomi, 6 parts of the Rhizoma coptidis, 10 parts of the Radix paeoniae alba, 10 parts of the Radix ophiopogonis, 20 parts of the red ochre, 20 parts of the calcined Ossa draconis, 20 parts of the calcined Concha ostreae, 10 parts of the Radix rehmanniae, 20 parts of the Bulbus fritillariae thunbergii, 10 parts of the Radix asparagi, 30 parts of the Radix astragali, 10 parts of the vinegar-quenched Carapax et Plastrum Testudinis, 10 parts of the Radix achyranthis bidentatae, 20 parts of the Fructus aurantii immaturus, 10 parts of the Pinelliae rhizoma praeparatum, and 10 parts of the Rhizoma atractylodis macrocephalae.

38. The preparation method according to claim 33, wherein a total volume of the calcined Ossa draconis, the calcined Concha ostreae, the vinegar-quenched Carapax et Plastrum testudinis, and the red ochre and a volume of the water are at a ratio of 1:2, while the first decoction is conducted at 100° C. to 125° C. for 30 min to 35 min in step 1).

39. The traditional Chinese medicine decoction according to claim 30, wherein a total volume of the calcined Ossa draconis, the calcined Concha ostreae, the vinegar-quenched Carapax et Plastrum Testudinis, and the red ochre and a volume of the water are at a ratio of 1:2, while the first decoction is conducted at 100° C. to 125° C. for 30 min to 35 min in step 1).

40. The traditional Chinese medicine decoction according to claim 30, wherein the remaining raw materials and the water are at a volume ratio of 1:1, while the second decoction is conducted at 100° C. to 125° C. for 30 min to 35 min in step 2).
